(19) United States
(12) Plant Patent Application Publication
(10) Pub. No.: US 2011/0030112 P1
(43) Pub. Date: Feb. 3, 2011

(54) POMEGRANATE TREE NAMED 'EMEK'

(50) ;

(75) Inventors: Doron Holland, Hasolelim (IL); Irit Bar-Ya'Akov, Qiryat Tivon (IL); Kamel Hatib, Arrabe (IL)

Correspondence Address: FOLEY AND LARDNER LLP;SUITE 500, 3000 K STREET NW, WASHINGTON, DC, 20007, US

(73) Assignee: STATE OF ISRAEL, MINISTRY OF AGRICULTURE & RURAL DEVELOPMENT, AGRICULTURAL RESEARCH ORGANIZATION

(21) Appl. No.: 12/462,241

(22) Filed: Jul. 31, 2009

(51) Int. Cl. A01H 5/00

(52) U.S. Cl. PLT/210

(57) ABSTRACT

A new and distinct pomegranate variety of Punica granatum named ‘EMEK’, particularly characterized by self-pollinating and self-fertile medium-sized tree with vigorous growth habit; good fruit productivity; large-sized, mature fruit, with uniform, dark red skin color that appears very early in fruit development, and red arils; and mature fruit which is of good eating quality (sweet-sour flavor and good juice production).

### LATIN NAME OF THE GENUS AND SPECIES OF THE PLANT CLAIMED

Punica granatum

### VARIETY DENOMINATION

‘EMEK’

### BACKGROUND OF THE INVENTION

The present invention relates to a new and distinct variety of pomegranate tree, botanically known as Punica granatum L. of the Lythraceae family, and hereinafter referred to by the variety denomination ‘EMEK’.

The new Punica granatum ‘EMEK’ is a product of a controlled breeding program conducted by the inventors, Doron Holland, Irit Bar-Ya'akov and Kamel Hatib in Yizre'el Valley, Israel. The objective of the breeding program was to develop a new Punica granatum variety with vigorous production of mature fruit with good eating quality.

Pomegranate seedlings of the inventors' proprietary pomegranate collection were collected from Yizre'el Valley, Israel, and planted in Yizre'el Valley, Israel. The new Punica granatum ‘EMEK’ originated from open pollination of an unknown, unpatented proprietary Punica granatum variety. The new Punica granatum ‘EMEK’ was discovered in 2003 and selected in 2006 by the inventors in a controlled environment in Yizre'el Valley, Israel.

Asexual reproduction of the new Punica granatum ‘EMEK’ by hardwood cuttings was first performed in February of 2006 in the Yizre'el Valley of Israel, and has demonstrated that the combination of characteristics as herein disclosed for the new variety are firmly fixed and retained through successive generations of asexual reproduction. The new variety reproduces true to type. Resulting trees of new Punica granatum were planted in 2007 in the Yizre'el Valley.

### BRIEF DESCRIPTION OF THE INVENTION

The following traits have been repeatedly observed and are determined to be unique characteristics of ‘EMEK’ which in combination distinguish this pomegranate tree as a new and distinct variety:
1. self-pollinating and self-fertile medium-sized tree with vigorous growth habit;
2. good fruit productivity;
3. early ripening with harvest of mature fruit in mid-August in Yizre'el Valley;
4. medium-sized, mature fruit, with dark pink to red skin color and red arils; and
5. mature fruit which is of good eating quality (sweet flavor and good juice production).

Of the many commercial varieties known to the present inventor, the most similar in comparison to the new Punica granatum ‘EMEK’ is Punica granatum ‘WONDERFUL’ (unpatented), in the following characteristics described in Table 1:

TABLE 1

| | | Comparison Variety |
|---|---|---|
| | New Variety | ‘WONDERFUL’ |
| Characteristic | ‘EMEK’ | (unpatented) |
| Ripening time | Early (mid August) | Late (beginning of October) |
| Mature fruit skin color | Dark pink to red | Red |
| Fruit size | Medium | Large |
| Taste | Sweet | Sweet-sour |

### BRIEF DESCRIPTION OF THE PHOTOGRAPHS

The accompanying photographs illustrate the overall appearance of the new Punica granatum ‘EMEK’ showing the colors as true as is reasonably possible with colored reproductions of this type. Colors in the photographs may differ slightly from the color values cited in the detailed botanical description, which accurately describe the color of ‘EMEK’.

FIG. 1 shows a side view perspective of a typical 2 year old shrub of ‘EMEK’.

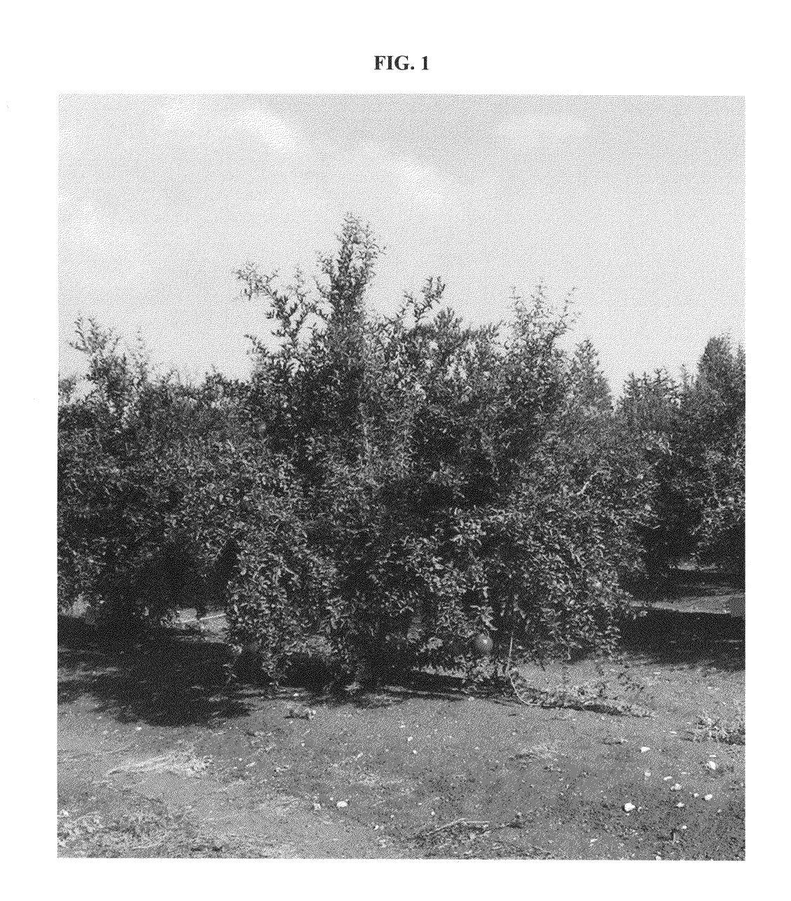

FIG. 2 shows a side view perspective of a typical trunk of ‘EMEK’.

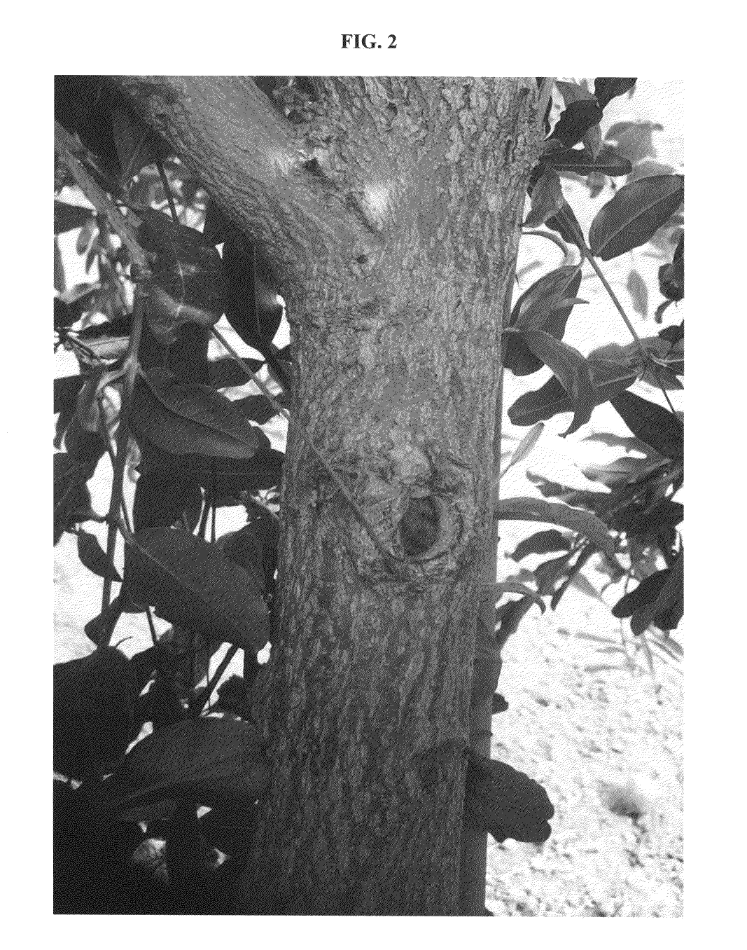

FIG. 3 shows a close-up view of typical fruit on a branch of ‘EMEK’.

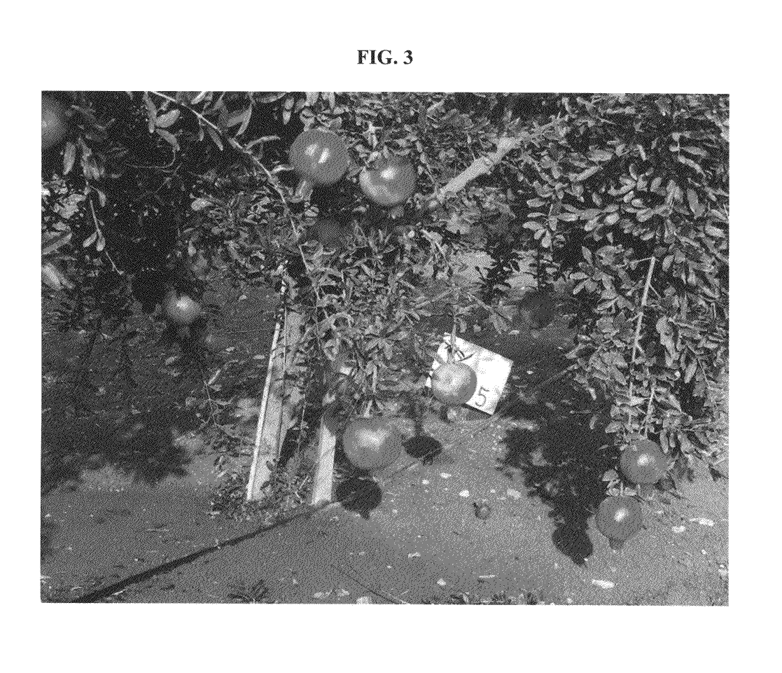

FIG. 4 shows a close-up view of typical leaves of ‘EMEK’.

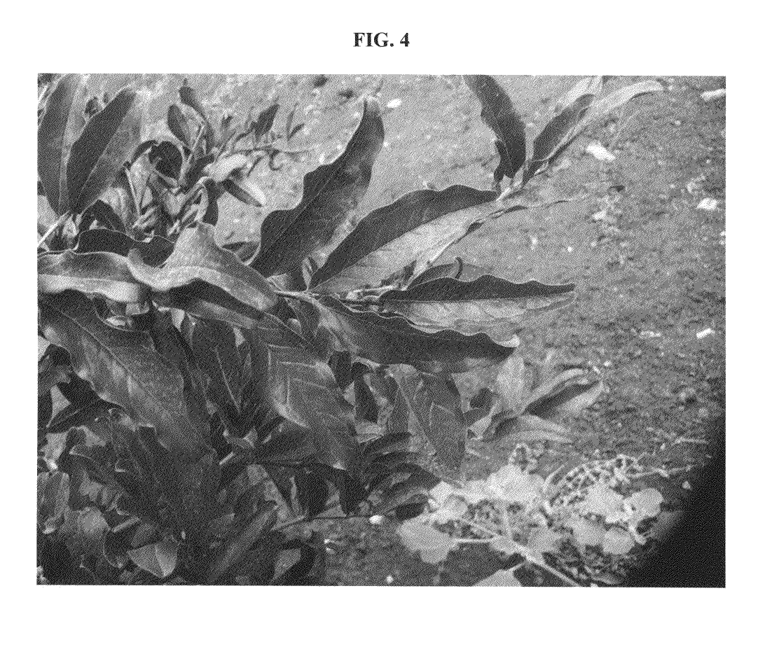

FIG. 5 shows a close-up view of typical hermaphrodite flower of ‘EMEK’.

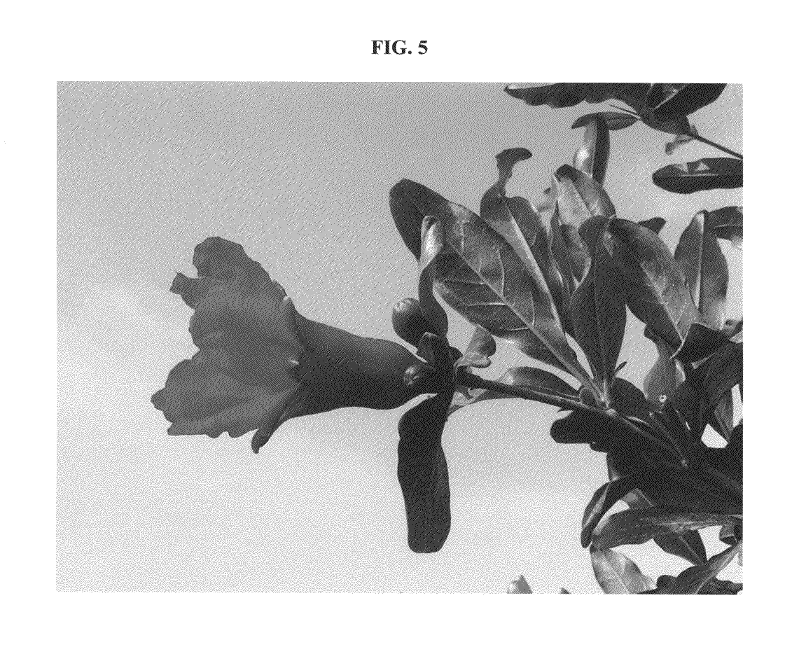

FIG. 6 shows a various close-up views of typical mature fruit of ‘EMEK’, illustrating the skin and arils color and fruit shape.

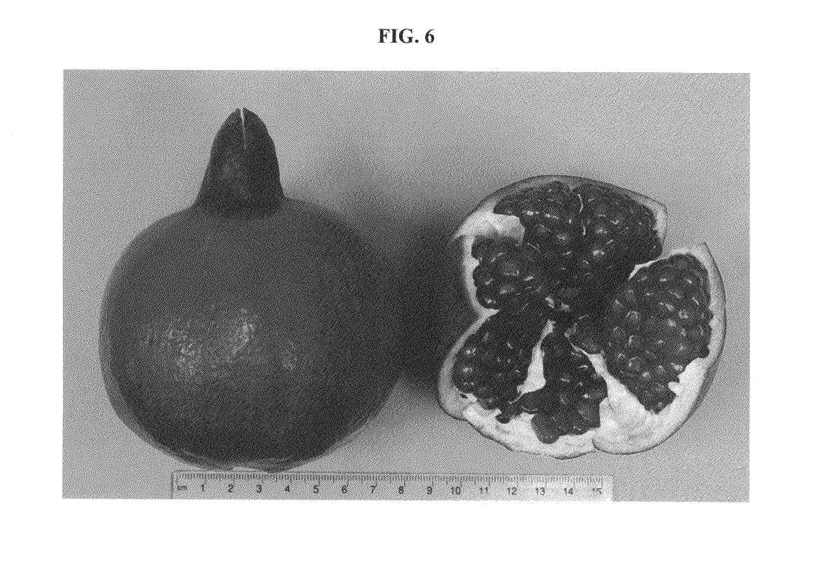

### DETAILED BOTANICAL DESCRIPTION

The new Punica granatum ‘EMEK’ has not been observed under all possible environmental conditions. The phenotype of the new variety may vary with variations in environment such as temperature, light intensity, and day length without any change in the genotype of the pomegranate tree.

The aforementioned photographs, together with the following observations, measurements and values describe trees of ‘EMEK’ as grown in the pomegranate farm in the Yizre'el Valley of Israel, under conditions which closely approximate those generally used in commercial practice. Seedlings of ‘EMEK’ were planted mainly in clay grumusol (vertisol) soil at an elevation of about 100 meters above sea level, at lat. 32° 42′ N, long. 35° 11′ E. Yizre'el Valley is characterized by a Mediterranean subtropical climate, with an average annual rainfall of about 580 mm concentrated from November through March. Mean diurnal minimum temperature in January is 6° C. (43° F.), and mean diurnal maximum temperature in July is 33° C. (91° F.).

Unless otherwise stated, the detailed botanical description includes observations, measurements and values based on two year old ‘EMEK’ trees grown in the pomegranate farm in Yizre'el Valley, Israel from 2006 to 2008. Quantified measurements are expressed as an average of measurements taken from a number of trees of ‘EMEK’. The measurements of any individual tree, or any group of trees, of the new variety may vary from the stated average.

Color references are made to The Royal Horticultural Society Colour Chart (RHS), 2001 edition, except where general colors of ordinary significance are used. Color values were taken under daylight conditions at approximately late morning in Yizre'el Valley, Israel.

All of the trees of ‘EMEK, insofar as they have been observed, have been identical in all the characteristics described below.
Classification:
   Botanical.—Punica granatum L.
Parentage: Open-pollination of unknown, unpatented, proprietary Punica granatum variety.
Propagation: Hardwood cuttings.
Growing conditions:
Light intensities: Full sunlight.
Fertilization: A level of about 250 Kg/ha N and 300 Kg/ha K per year and about 60 Kg/ha P every three years.
Growth regulators: Not used.
Pruning or trimming requirements: Winter pruning and removal of suckers during summer.
Tree:
Age: Observed trees were two years old.
Vigor: Good.
Form: Upright and spreading, medium sized tree with one trunk.
Branching habit: Main branches angle is up to 45° with respect to trunk if allowed to grow naturally.
Production: Good.
Size at maturity:
   Height.—About 2.5 m to 2.6 m.
   Spread.—About 2.3 to 2.4 m.
Trunk:
   Height (up to leaders).—About 50 cm to 60 cm.
   Diameter (15 cm above ground).—About 26.0 cm±0.6 cm.
   Texture.—Rough.
   Bark color.—Brown, RHS N200C, and grey-brown, RHS 199D.
   Trunk Lenticels.—None.
Branches:
   Number per tree.—About 3 or 4 depending on pruning.
   Length.—2 to 3 m.
   Diameter (about 10 cm above branching).—About 17.6 cm±1.0 cm.
   Growth per season.—On average, branches grow up to 2 m per growing season, and then new branchlets form, radiating from the older, primary branch.
   Surface texture.—New Growth: Semi-rough Mature: Rough.
   Color.—New Growth: Grey-brown, RHS 199A Mature: Greyed-green, RHS 191A.
   Internode length.—About 60 mm to 70 mm.
   Internode diameter.—About 3 mm to 4 mm.
   Branch spines.—Length: About 3 mm to 5 mm Width: About 1 mm Color: Brown Red Greyed-orange, RHS 175A Branch lenticels: None.
Spur: Present.
   Distance between each spur.—Varies.
   Thickness of each spur.—About 3 mm to 4 mm.
   Number of fruit per spur.—About 1 or 2.
Foliage:
Arrangement: Opposite, pairs alternately crossing at right angles, petiolated.
Lamina:
   Size (fully developed lamina).—Length: About 6.3 cm±0.2 cm Width: About 2.2 cm±0.1 cm.
   Overall shape.—Oblanceolate.
   Base shape.—Acuminate.
   Apex shape.—Obtuse.
   Margin.—Entire.
   Texture.—Upper surface: Smooth Under surface: Smooth.
   Color (mature leaves).—Upper surface: Green, RHS 137A Under surface: Green, RHS 137C.
   Color (immature leaves).—Upper surface: Green RHS 137A Under surface: Green, between RHS 144A and RHS 144B.
Venation:
   Type.—Pinnate.
   Color.—Green midrib, RHS 137C, about 1 mm in thickness.
Petiole:
   Length.—About 5.0 mm±0.2 mm.
   Diameter.—About 1.0 mm.
   Color.—Green, RHS 137C.
Stipule: Exstipulate.
Glands: None.
Inflorescence:
Blooming time: Full bloom on about beginning of May in Yizre'el Valley, Israel.
Blooming period: About one month.
Fragrance: None.
Inflorescence:
   Length.—Up to 15 cm.
   Type.—Hermaphrodite flowers are born single or clustered. The flowers can be found on the same plant in various shapes and size. There are vase shaped flowers with fully developed reproductive organs and bell shaped flowers which have undeveloped reproductive organs, and between these two types there are various flower shapes and stages of.
Flower diameter: About 45 mm to 50 mm.
Flower depth: About 32 mm to 42 mm.
Buds:
   Number per spur.—About 1 or 2.
   Shape.—Obovate.
   Length.—About 15 mm.
   Width.—About 8 mm to 10 mm.
   Color.—Dark orange-red, closest to RHS 34A.
   Scales.—Typical to species, not distinguishing.
Calyx:
   Depth.—50 mm to 55 mm.
   Diameter (funnel).—About 31 mm to 43 mm.
   Attitude.—Semi-erect to horizontal.
   Form.—Narrow funnel, which acute apex and thickened at tip.
   Calyx lobes length.—About 10 mm to 12 mm.
   Calyx lobes width.—About 7 mm to 9 mm.
   Color.—Dark orange-red, closest to RHS 33B.
Petals:
   Number per flower.—About 5 to 7.
   Size.—Length: About 20 mm to 23 mm Width: About 17 mm to 20 mm.
   Overall shape.—Elliptic.
   Apex shape.—Rounded.
   Base shape.—Acute.
   Texture (upper surface).—Smooth.
   Texture (lower surface).—Smooth.
   Margin.—Entire, undulated.
   Mature Color.—Upper surface: Orange-red, RHS33B Under surface: Orange-red, RHS 33B.
Sepals:
   Number per flower.—About 5 to 7.
   Size.—Length: About 10 mm to 12 mm Width: About 7 mm to 9 mm.
   Overall shape.—Triangular.
   Apex shape.—Very acute.
   Base shape.—Fused.
   Texture (upper surface).—Smooth.
   Texture (lower surface).—Smooth.
   Margin.—Entire.
   Mature Color.—Outer surface: Orange-red, RHS 33B Inner surface: Orange-red, RHS 33A.
Pedicle: Flowers are sessile, no pedicle.
Fruit:
Keeping quality: The fruit keeps well on the tree. It can be stored in cold temperature conditions at above 7° C. for about 4 weeks in good eating quality.
Maturity when described: Ripe for commercial harvesting and shipment; about mid-August in Yizre'el Valley, Israel.
Maturity period after full bloom: About 3½ months after full bloom about beginning of May in Yizre'el Valley, Israel.
Date of first picking (harvest): About August 15^{th} in Yizre'el Valley, Israel in 2008.
Type: Berry.
General shape: Rounded, nearly uniform, symmetrical.
Apex shape: Prominent crown of calyx segments at apex.
Base shape: Symmetrical base, slightly flattened near stem end.
Suture: None.
Average weight: About 380 g.
Fruit Size:
   Average height (axial plane, without crown).—About 86 mm±1.0 mm.
   Average diameter (widest pint, right angle to axial plane).—About 273 mm±3.0 mm.
Crown:
   Height.—About 34 mm±1.0 mm.
   Circumference.—About 89 mm±2.0 mm.
   Spikes.—Number of about 6 to 7 per fruit; position half-closed.
Rind:
   Thickness.—Medium.
   Texture.—Smooth and shiny.
   Tendency to crack.—None.
   Ground color.—Pinkish-orange.
   Blush color.—Dark pink to red.
   Percentage of skin surface with overcolor color.—Uniformly spread over entire rind.
Pulp membrane:
   Thickness.—Thin.
Arils:
   Texture.—Smooth and shiny.
   Length.—About 10 mm to 13 mm.
   Diameter.—About 7 mm to 9 mm.
   Weight (per aril).—About 0.36 g.
   Volume (per aril).—About 0.32 cm³.
   Color at maturity.—Dark red, RHS 45A.
   Separation.—Easy.
   Septum.—Thin, transparent.
   Ripening.—Uniform.
   Fibers.—Not noticeable.
   Aroma.—None.
   Eating quality.—Very good.
   Juice production.—Good.
   Flavor.—Sweet.
Seeds:
   Overall shape.—Ovate.
   Apex shape.—Apex.
   Base shape.—Rounded.
   Length.—About 8 mm.
   Dry weight (per seed).—About 0.024 g.
   Dry volume (per seed).—About 0.05 cm³.
   Hardness.—Relatively soft.
   Color (dry).—White, RHS 155C.
Reproductive organs:
Androecium:
   Stamen.—Number per flower: Numerous.
   Anther.—Length: About 7 mm to 9 mm Color: Yellow, RHS 4A.
   Filaments.—Length: About 7 mm to 9 mm Color: Dark orange, RHS 33A Pollination Requirements: None, ‘EMEK self-fertile and self-pollinator.
Gynoecium:
   Pistils.—Quantity: One. Length: About 5 mm to 7 mm Diameter: About 1 mm Color: Dark orange, RHS 32B.
   Stigmas.—Size: Very small Color: Greenish-yellow, RHS 1A.
   Ovary.—Shape: Conic Length: About 10 mm to 13 mm. Diameter: About 13 mm to 20 mm. Color: Orange, RHS 25C Pubescence: None.
Market: Fresh market and food, juice and dietary supplement manufacturing.
Usage: Fruit and extract.
Disease/pest resistance: No atypical resistance has been noted.
Disease/pest susceptibility: No atypical resistance has been noted.
Winter hardiness: Tolerant to temperatures down to −3° C. without observed damage to wood and buds of dormant pomegranate trees, under prevailing local conditions.

What is claimed is:

1. A new and distinct pomegranate variety of Punica granatum named ‘EMEK’, as illustrated and described herein.
